(19) United States
(12) Patent Application Publication
(10) Pub. No.: US 2008/0121248 A1
(43) Pub. Date: May 29, 2008

(54) Laser Decontamination of the Surface of a Profiled Part

(75) Inventors: Bernard Turco, Orsan (FR); Marc Fisher, Montelimar (FR)

Correspondence Address: THELEN REID BROWN RAYSMAN & STEINER LLP, P. O. BOX 640640, SAN JOSE, CA, 95164-0640, US

(73) Assignee: Compagnie Generale Des Matieres Nuleaires, Velizy Villacoublay (FR)

(21) Appl. No.: 11/793,109

(22) PCT Filed: Dec. 12, 2005
(86) PCT No.: PCT/FR05/51071

§ 371 Date: Jun. 14, 2007

(30) Foreign Application Priority Data
Dec. 14, 2004 (FR) 0452967

(51) Int. Cl. B08B 7/00; B23K 26/14; B23K 26/36

(52) U.S. Cl. 134/1; 422/285

(57) ABSTRACT

The invention concerns laser decontamination of the surface of a shaped component (3). Said decontamination comprises the following steps: successively impinging parts of the surface of the shaped component (3) with pulsed laser beams of wavelengths in the ultraviolet range and enabling the surface layer of the shaped component (3) to be ablated in the form of particles, the laser beams being distributed so as to cover simultaneously each treated part; and recuperating by suction said particles.

### TECHNICAL FIELD

The invention relates to decontamination (in particular radioactive decontamination) of the surface of a profiled part by means of laser beams.

By profiled part, is meant a part with a relatively large length relatively to its cross-section, this section remaining constant. This may be a metallurgical product obtained for example by hot-rolling or cold-drawing. This may also be (and the present invention will more specifically treat this case) nuclear fuel pencils.

More specifically, the invention is related to removal of material from the surface of a profiled part, consisting of submitting the surface of the part to the impact of a pulsed laser beam, emitting in the ultraviolet, and of simultaneously sucking up through a filter, the removed material from the surface of the profiled part.

However, the invention may be applied in all fields of the industry in which it is necessary to remove a material layer from the surface of such a part.

### STATE OF THE PRIOR ART

A nuclear fuel pencil (or needle) is formed by a column of fissile material contained in a sheath. This sheath is generally a metal tube provided with welded plugs at its ends. For example, the fissile material is introduced into the sheath as tablets.

Introducing the tablets in their sheath has a potential risk of contamination of the outer surface of the pencil, in particular of the end of the pencil brought closer to the “nose” of the sheathing.

Decontamination of the pencils proves to be necessary for the following reasons. Technical specifications require that residual contamination of the pencils be less than a certain threshold: 0.4 Bq/dm² as labile contamination and 83 Bq/dm² as fixed contamination. These thresholds are justified by the handling of the pencils in the factory, out of confinement.

Contamination present on the pencils consists of submicron particles stemming from the constitutive material of the fuel tablets. If the fissile material consists of MOX (Mixed OXide) tablets, these particles are in UO₂, in PuO₂ and in their mixture. The sheath of the pencils should have good mechanical strength and should retain it up to the end of the life of the fuel. This feature is notably shown by the retention of the original aspect of the sheath: white, bright and uniform. It should notably remain ductile so that repeated deformations, due to variations of temperature and pressure in the reactors, do not cause cracking. They may advantageously be made in a zirconium alloy which is called Zircaloy®.

The few laser cleaning projects within the scope of nuclear decontamination, published in the public press, were achieved in the United States and in France. At the beginning of the eighties, the US Department of Energy (DOE) proposed the use of power lasers for decontaminating nuclear facilities. The main published results were obtained between 1992 and 1996, by a team from the Ames Laboratory within the scope of the <<Ames Laser Decontamination Project>>. They used both a 100 W (248 nm, 27 ns) KrF excimer laser and a Q-switched (1064 nm, 100 ns) Nd: YAG laser for studying the displacement of radioactive oxide on metal surfaces (aluminium, steel, copper, wire). The effectiveness of this ablation method proved to be larger with a larger ionization potential of the ambient gas. Cylindrical objectives focusing the beam were used in order to well cover the surface and to reduce redeposition of particles. These experiments were conducted with false contaminants, radioactive samples and with equipment of large dimensions stemming from nuclear installations. Efficiency of the decontamination was generally sufficient for these specific applications. The Nd: YAG laser was proposed in order to develop a prototype as its wavelength (1064 nm) may be effectively transmitted by conventional optical fibers.

Other projects were focussed on decontaminating concrete. In this case, the pollutants diffuse in depth and decontamination may be obtained by ablation of a thick layer of concrete (several millimetres). For these experiments, power infrared lasers such as CO₂ or Nd:YAG lasers are used. For this purpose, a prototype of a cleaning laser based on Nd:YAG, was then designed in the Argonne National Laboratory and it was probably tested for decommissioning a nuclear reactor. Recently, a chemical laser called COIL (Chemical Oxygen Iodine Laser), initially designed as a military laser weapon, operation at more than 1 kW, has been proposed for dismantling nuclear installations.

Since 1999, French R & D has been following the same route. Experiments were carried out with Nd:YAG and excimer lasers. EP-A-0 520 847 discloses a laser-based tool for decontaminating a steam generator from nuclear installations. FR-A-2 774 801 discloses a method and an installation for decontaminating nuclear fuel pencils by means of a laser beam. The results of the techniques exposed by these documents have not been disclosed.

The LEXDIN prototype was designed and tested by the <<Commissariat a l'Energie Atomique >> (French nuclear agency) in the case of the decontamination of a plexiglas chamber (cf. J. R. COSTES et al., <<Decontamination by ultraviolet laser: the LEXDIN Prototype>>, CEA, 1996). This prototype uses a XeCl laser and mirrors for transporting the laser beam.

A comparative study was conducted between the Nd:YAG laser and the KrF excimer laser for dry and hot cleaning of contaminated samples (steel, Inconel) stemming from a steam generator. Under the air atmosphere, there was a slight influence of the wavelength of the laser. With a liquid film confining the plasma, it would be possible to increase the efficiency of the cleaning (cf. FR-A-2 700 882). As compared with dry cleaning, the decontamination factor was 30 times larger with a water film, 85 times larger with a film of 0.5 M nitric acid and 650 times larger with a film of 5 M nitric acid.

Other decontamination projects by means of a laser beam were undertaken by applying a XeCl excimer laser, a Nd:YAG laser (6 ns) and a flash lamp xenon laser (200 ms). However, it seems that no laser decontamination system was utilized inside a nuclear installation for cleaning and dismantling.

### DISCUSSION OF THE INVENTION

The present invention proposes a laser decontamination method and a device applying this method and utilizable inside a nuclear installation.

A first object of the invention consists of a method for laser decontamination of the surface of a profiled part, characterized in that it consists of:
successively submitting portions of the surface of the profiled part to the impact of pulsed laser beams with wavelengths located in the ultraviolet and with which the surface layer of the profiled part may be ablated as particles, the laser beams being distributed so as to simultaneously cover each treated portion;
recovering said particles by suction.

Advantageously, said laser beams are obtained by dividing a main laser beam and they are of equal energy. Also advantageously, said divided laser beams each travel along a trajectory of equal length from their division with the main laser beam.

Preferably, recovery of said particles is carried out by filtering the particles.

A second object of the invention consists of a device for laser decontamination of the surface of a profiled part, characterized in that it comprises:
a treatment chamber provided with a hole for letting through said profiled part so as to successively bring portions of the surface of the profiled part into a treatment area, the treatment chamber further comprising optical paths for providing the passage for pulsed laser beams with wavelengths located in the ultraviolet and with which the surface layer of the profiled part may be ablated as particles, the optical paths being distributed so as to simultaneously cover each treated portion of the surface of the profiled part, the treatment chamber also comprising suction means for recovering said particles;
means for conveying said laser beams up to the treatment chamber.

Advantageously, the suction means comprise means for introducing a gas jet located so that the gas transits through the optical paths before reaching the treatment area where it is then directed towards a chamber for collecting said particles.

Advantageously, the collection chamber comprises means for filtering said particles. The optical paths may comprise means for retaining said particles, intended to retain ablated particles which may flow in the counter-direction to the gas jet.

Advantageously, the optical paths are conduits provided with sealing windows towards the outside of the device and providing the passage for the laser beams.

Advantageously, the means for transporting said laser beams comprise means for dividing a main laser beam in order to obtain said laser beams of equal energy. These means for dividing the main laser beam may comprise bundles of optical fibers. They may comprise semi-reflecting mirrors and total reflection mirrors. Preferably, the means for dividing the main laser beam are means providing laser beams traveling over equal trajectories.

### SHORT DESCRIPTION OF THE DRAWINGS

The invention will be better understood and other features and particularities will become apparent upon reading the description which follows, given as a non-limiting example, accompanied with appended drawings wherein:

FIG. 1A is a cross-sectional view of the treatment chamber of a device for decontaminating a profiled part, according to the present invention;

FIG. 1B is a view along the plane BB of FIG. 1A;

FIG. 2 is a first block diagram of the division of main laser beam into four decontamination laser beams and which may be applied in the device of the present invention;

FIG. 3 is a second block diagram of the division of a main laser beam into four decontamination laser beams and which may be applied in the device of the present invention;

FIG. 4 is a third block diagram of the division of main laser beam into four decontamination laser beams and which may be applied in the device of the present invention;

FIG. 5 is a fourth block diagram of the division of a main laser beam into four decontamination laser beams and which may be applied in the device of the present invention.

### DETAILED DISCUSSION OF PARTICULAR EMBODIMENTS

Upon irradiating a surface with a pulsed laser, a portion of the laser energy is absorbed by the material and this generates a temperature gradient in the thickness of the material which depends on its absorptivity at the wavelength of the laser. Three processes are mainly at the origin of the ablation phenomenon: a heat mechanism, a mechanical mechanism, and a photochemical mechanism. These mechanisms are closely related but depending on the nature of the substrate on the wavelength and on the pulsed duration of the laser, one of them becomes predominant in the ablation process.

The absorptivity of metal material is all the more significant as the wavelength of the laser is short. The very energetic photons (4-8 eV) of excimer lasers are absorbed by a small thickness of the material (a few nanometers) and the heat diffusion length is typically of the order of one micrometer. They transfer their energy to the electrons of the material which by deexcitation will cause heating of the irradiated material. When the temperature of the volume which has absorbed the laser energy is higher than the vaporization temperature of the material, the ablation process is initiated. This ablation process of thermal origin is characterized by the formation of a plasma consisting of electrons and neutral and ionized species from the irradiated substrate and the ambient gas. This plasma is propagated perpendicularly to the surface of the material. The strong UV absorptivity of the substrate only allows the temperature to be raised in a small volume, and therefore to ablate only a thin layer of the material while limiting the deeper thermal effect. The short pulse durations of excimer lasers between 10 and 30 ns, further limit heat diffusion and therefore the thermal effects inside the non-ablated volume.

For low fluencies, the temperature reached at the surface is not sufficient for causing vaporization of the material, but energy absorption by the surface, and/or the compounds (particles, layers) which are deposited thereon, induces a fast increase of their temperature. This will cause sudden expansion of the heated volumes and, if the acceleration which is then transmitted to the compounds deposited on the surface is sufficient, they may be ejected from the latter. This ablation process of mechanical origin does not change the structure and the morphology of the substrate. It is predominant during the cleaning of microparticles by laser. It may be applied via a dry route by directly irradiating the surface with the laser beam or via a wet route by depositing a liquid film on the surface before irradiation, and in this case, it is the ablation of the film which will cause ejection of the particles. It is at the origin of the removal of oxide particles. It is effective because the materials (substrate and particles) strongly absorb UV laser radiation.

The processes induced by irradiating a surface with an ultraviolet beam may also be of photochemical origin when the energy of the photons (4-8 eV) is larger than the binding energies of the irradiated compounds. This ablation process of photochemical origin generally occurs for substrates such as polymers. Main applications are the stripping of paint and engraving of polymer materials.

The invention relates to a method for removing material from the surface of a profiled part, consisting of submitting the surface of the profiled part to the impact of a pulsed laser beam emitting in the ultraviolet and of simultaneously sucking through a filter the material removed from the surface of the profiled part. This is a mechanical mechanism which is applied by the invention.

A device for decontaminating profiled parts, according to the present invention, comprises a treatment chamber maintained at a negative pressure and a system for shaping a laser beam.

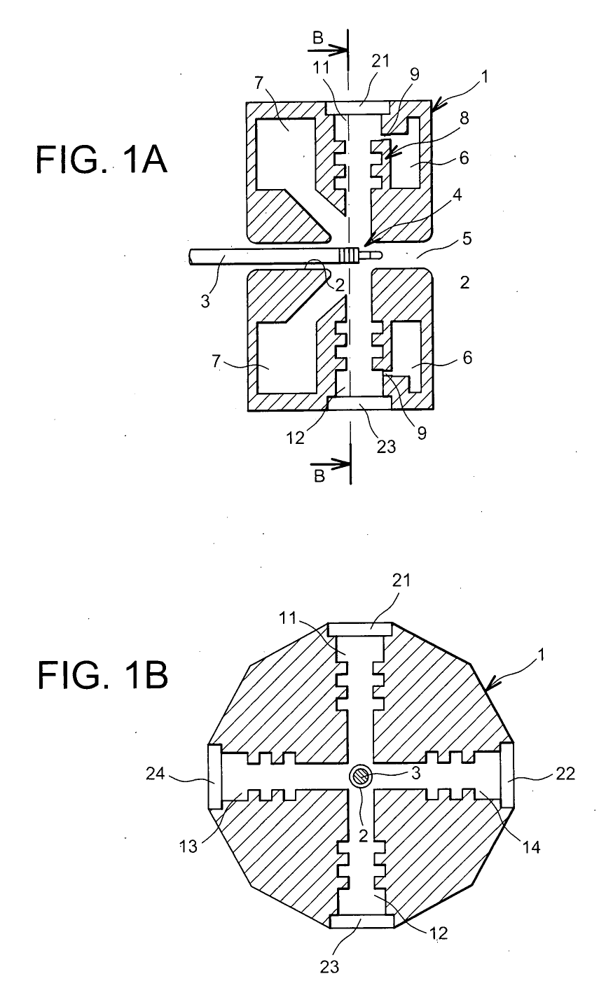

The treatment chamber 1 is illustrated in FIGS. 1A and 1B. It is provided with a central hole 2 through which a profiled part 3 to be treated may be introduced. The treatment of the profiled part 3 is performed in the area referenced as 4 in FIG. 1A. The area 4 corresponds to the junction of four optical paths 11, 12, 13 and 14 with equal lengths and positioned along two orthogonal axes. The optical paths 11, 12, 13 and 14 are blocked by windows 21, 22, 23 and 24, respectively, which provide the seal with the outside of the treatment chamber 1 and which allow the passage of the laser beam required for the decontamination.

The profiled part to be decontaminated is introduced into the treatment chamber 1 through the central hole 2 provided with a ball cage not shown, in order to ensure perfect centering of the part. The profiled part 3 advances through the chamber at a given rate. With the parameters, advance rate of the part and frequency of the laser beams, it is possible to obtain the number of laser pulses required for the decontamination.

One or more vacuum pumps enable a negative pressure to be generated inside the treatment chamber, so that the contaminants may be sucked up by means of a set of VHE filters (very high efficiency filters).

Air penetrates into the decontamination chamber in two ways: through the exit port 5 of the profiled parts and through an annular chamber 6 located close to the windows and communicating with the optical paths via channels 9. With this arrangement, it is also possible to avoid redeposition of the contaminants on the windows, which may affect the efficiency of the decontamination. Under the impact of the laser beams on the surface of the profiled part 3, particles are sucked up because of the negative pressure existing inside the decontamination chamber. These particles are directed, according to an angle of 45° relatively to the axis of the incident laser beam, into an annular collection chamber 7 so as to be captured subsequently by the VHE filters. To be effective, suction is performed as close as possible to the treatment area 4. On the trajectories of the laser beams, i.e., in the optical paths 11, 12, 13 and 14, indentations 8 are laid out in order to prevent the extracted particles from being deposited on the inner surface of the windows 21, 22, 23 and 24.

The treatment laser beams advantageously stem from the division of a main laser beam. The distance traveled by each of these incident laser beams is identical from the division point of the main laser beam up to the treatment area, which provides perfect treatment homogeneity. With several laser beams, it is possible to simultaneously illuminate the periphery of the profiled part. In the example described here, the divided beams are four in number but the number of divided beams may be different.

The main laser beams may be conveyed by a bundle of optical fibers. The divided or secondary laser beams are then generated by dividing the main bundle of optical fibers.

The main laser beam may be conveyed by reflection on mirrors. The secondary laser beams are generated by dividing the main laser beam by means of mirrors and beam splitters.

The circuit for sucking up extracted particles is defined with rather small passage sections in order to maintain high flow rates (gas+particles) and to thereby limit deposits of particles which may adhere to surfaces through a mechanical effect or through Van Der Waals' forces, the potential of which may reach 2-5 eV according to the materials.

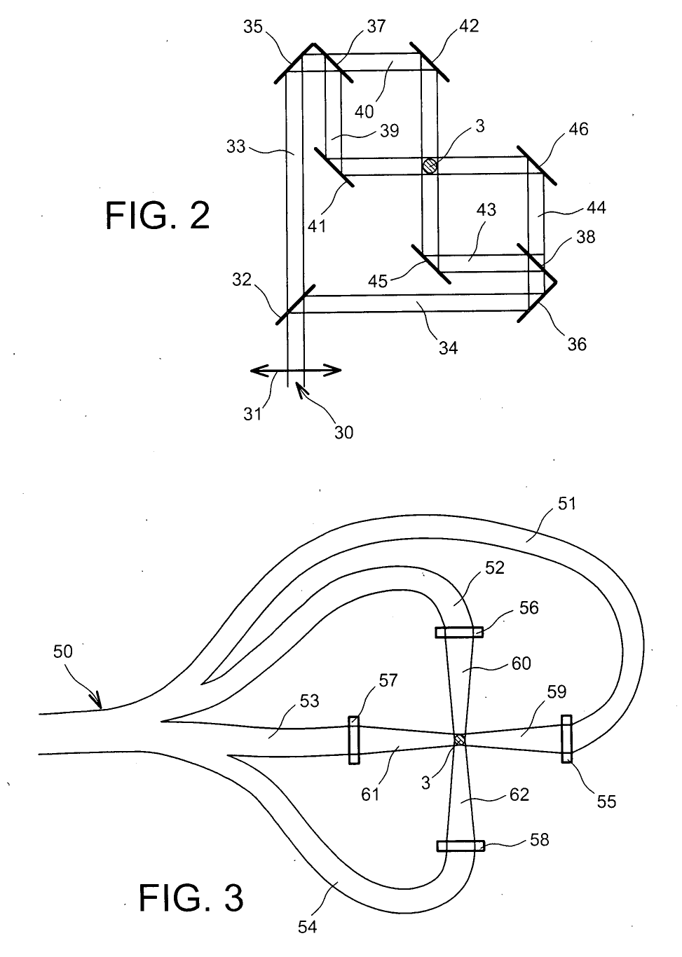

FIGS. 2-5 are different block diagrams of the division of a main laser beam into four decontamination beams and which may be applied in the device of the present invention.

In the diagram of FIG. 2, a main laser beam 30, shaped by a lens 31, is sent onto a 50%-reflecting mirror 32. The mirror 32 divides the main laser beam into two beams 33 and 34 which are directed towards 100%-reflecting mirrors, 35 and 36, respectively. The beams 33 and 34 are then reflected towards 50%-reflecting mirrors, 37 and 38, respectively The beam 33 is then divided into two secondary beams 39 and 40 which are sent back, by the 100%-reflecting mirrors 41 and 42, respectively, towards the surface of the profiled part 3. The beam 34 is itself also divided into two secondary beams 43 and 44 which are sent back, by the 100%-reflecting mirrors 45 and 46, respectively, towards the surface of the profiled part 3. The four secondary beams 39, 40, 43 and 44 are of equal energy and have traveled the same distance up to the profiled part 3.

In the diagram of FIG. 3, a main laser beam is conveyed via a main bundle of optical fibers 50. The main bundle of optical fibers 50 is separated into four bundles of secondary fibers 51, 52, 53 and 54 with equal lengths (unlike what is shown in the diagram of FIG. 3 which is a block diagram). The bundles of secondary fibers 51, 52, 53 and 54 are brought in front of optics for reshaping the beam, 55, 56, 57 and 58 respectively, which provide beams 59, 60, 61 and 62, respectively, with equal energy and incident on the surface of the profiled part 3.

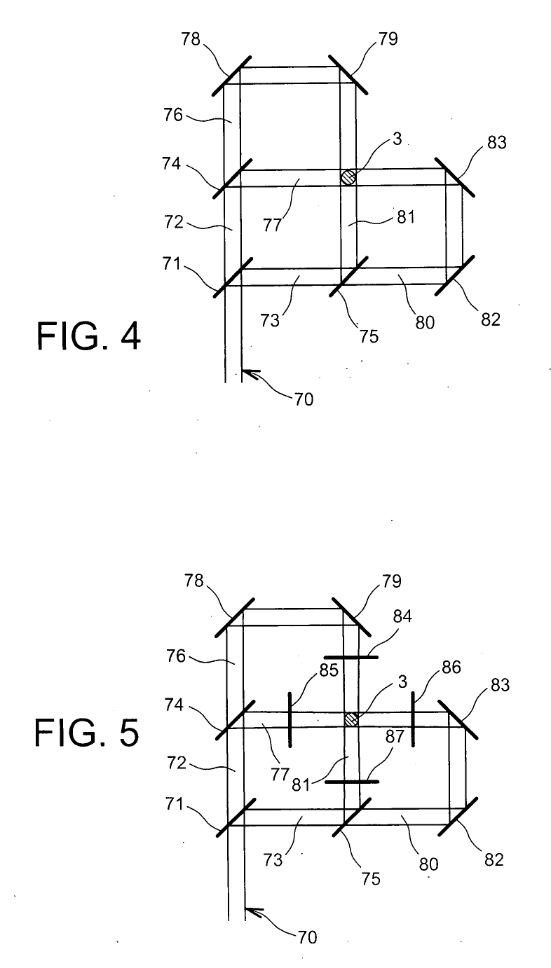

In the diagram of FIG. 4, a main laser beam 70 is sent onto a 50%-reflecting mirror 71. The mirror 71 divides the main laser beam into two beams 72 and 73 which are directed towards other 50%-reflecting mirrors, 74 and 75, respectively. The beam 72 is then divided into two secondary beams 76 and 77 which are directed towards the profiled part 3, directly for the secondary beam 77 and via two 100%-reflecting mirrors 78 and 79 for the secondary beam 76. The beam 73 is itself also divided into two secondary beams 80 and 81 which are directed towards the profiled part 3, directly for the secondary beam 81 and via two 100%-reflecting mirrors 82 and 83 for the secondary beam 80. The profiled part 3 is then submitted to the impact of four parallel beams with equal energy.

In the diagram of FIG. 5, the same structure as the diagram of FIG. 4 is again found. In the diagram of FIG. 5, focussing optics 84, 85, 86 and 87 have been added on the trajectory of the secondary beams, 76, 77, 80 and 81, respectively, incident on the surface of the profiled part 3. The profiled part 3 is then submitted to the impact of four converging beams with equal energies.

1. A method for laser decontamination of the surface of a profiled part (3), characterized in that it consists of:
successively submitting portions of the surface of the profiled part (3) to the impact of pulsed laser beams (39, 40, 43, 44) with wavelengths located in the ultraviolet and with which the surface layer of the profiled part may be ablated as particles, the laser beams being distributed so as to simultaneously cover each treated portion;
recovering said particles by suction.

2. The method according to claim 1, wherein said laser beams are obtained by dividing a main laser beam (30) and are of equal energy.

3. The method according to claim 2, wherein said divided laser beams each travel over a trajectory of equal length from their division with the main laser beam.

4. The method according to claim 1, wherein the recovery of said particles is carried out by filtering the particles.

5. A device for laser decontamination of the surface of a profiled part, comprising:
a treatment chamber provided with a passage hole for said profiled part with which portions of the surface of the profiled part may be successively brought into a treatment area, the treatment chamber further comprising optical paths providing the passage for pulsed laser beams with wavelengths located in the ultraviolet and with which the surface layer of the profiled part may be ablated as particles, the optical paths being distributed so as to simultaneously cover each treated portion of the surface of the profiled part, the treatment chamber also comprising suction means for recovering said particles;
means for conveying said laser beams up to the treatment chamber.

6. The device according to claim 5, wherein the suction means comprise means for introducing a gas jet located so that the gas transits via the optical paths before reaching the treatment area where it is subsequently directed towards a chamber for collecting said particles.

7. The device according to claim 5, wherein the collection chamber comprises means for filtering said particles.

8. The device according to claim 6, wherein the optical paths comprise means for retaining said particles, intended to retain ablated particles which may flow in the counter-direction to the gas jet.

9. The device according to claim 5, wherein the optical paths are conduits provided with sealing windows towards the outside of the device and providing the passage for the laser beams.

10. The device according to claim 5, wherein the means for transporting said laser beams comprise means for dividing a main laser beam in order to obtain said laser beams with equal energy.

11. The device according to claim 10, wherein the means for dividing the main laser beam comprise bundles of optical fibers.

12. The device according to claim 10, wherein the means for dividing the main laser beams comprise semi-reflecting mirrors and total reflection mirrors.

13. The device according to claim 10, wherein the means for dividing the main laser beam are means providing laser beams traveling over equal trajectories.

14. The device according to any of claim 7, characterized in that the optical paths comprise means for retaining said particles, intended to retain ablated particles which may flow in the counter-direction to the gas jet.
